(19) United States
(12) Patent Application Publication
(10) Pub. No.: US 2008/0228224 A1
(43) Pub. Date: Sep. 18, 2008

(54) Device For Securing A Surgical Suture To A Bone

(76) Inventors: Michael Sauer, Tuttlingen (DE); Sebastian Frey, Villingen-Schwenningen (DE)

Correspondence Address: ST. ONGE STEWARD JOHNSTON & REENS, LLC, 986 BEDFORD STREET, STAMFORD, CT, 06905-5619, US

(21) Appl. No.: 12/047,121

(22) Filed: Mar. 12, 2008

(30) Foreign Application Priority Data
Mar. 13, 2007 (DE) 10 2007 013 426.8

(51) Int. Cl. A61B 17/04

(52) U.S. Cl. 606/233; 606/300

(57) ABSTRACT

A device for securing a surgical suture to a bone comprises a first element, which can be introduced into a bone, and a second, movable element, which can be introduced into said bone in such a way that it meets said first element. A channel being formed in said bone as a result of introducing said first and said second elements into that bone. One of said two elements carries a surgical suture. A suture catcher is arranged within that element of the two elements not carrying said suture, by means of which suture catcher said suture can be taken hold of and said suture can be withdrawn via said element not carrying said suture.

### BACKGROUND OF THE INVENTION

This invention relates to a device for securing a surgical suture to a bone.

A device of this kind is known from U.S. Pat. No. 6,843,796 B2.

The known device for securing a surgical suture to a bone, has a first element, which can be introduced into a bone, and a second, movable element, which can be introduced into the bone in such a way that it meets the first element, as a result of which a channel is formed in the bone. One of the two elements carries a surgical suture in such a way that the suture can be taken hold of after the elements have met.

By means of such a device, an arc-shaped channel can be formed in a bone. Thereafter, the device is removed from the bone, and a surgical suture is guided through the channel with the aid of a suture pusher. With the free ends of the suture protruding from both ends of the channel, it is possible for a bone fragment, a torn tissue or an implant-holding suture to be fixed to the bone.

The device mentioned at the outset comprises two curved elements that can be moved towards each other. A channel extends through each element. The elements are shaped like the jaws of a pair of pliers. A sharp probe, on which a surgical suture is fixed, is arranged on a distal end of one of the elements. The surgical suture is thus introduced by this movable element into the bone.

To form the channel, the two elements are driven into the bone, specifically until the probe on which the suture is fixed meets a distal end of the second element in the inside of the bone. The probe travels a short distance into the channel of the second element. When the second element is pulled back, the probe is intended to remain therein, and the suture should be pulled together with the probe out of the bone by means of the second element.

A disadvantage of said device is that the probe can come loose from the second element and remains in the channel when said element is pulled back. The second element then has to be driven in once more in order to catch the lost probe again. It can also happen that the transfer of the probe from the first element to the second element does not function, and that it therefore remains on the first element. The incorrect function is only noticed when the second element is withdrawn from the bone, since the transfer site lies in the inside of the bone and cannot therefore be seen from outside. If the elements have to be driven in several times, this causes unnecessary trauma.

It is therefore an object of the present invention to develop a device of the kind mentioned at the outset in such a way that the suture transfer can be carried out with a high success rate, as far as possible a 100% success rate.

### SUMMARY OF THE INVENTION

According to the invention, this object is achieved by a device for securing a surgical suture to a bone, comprising a first element which can be introduced into a bone, a second, movable element, which can be introduced into said bone in such a way that it meets said first element, a channel being formed in said bone as a result of introducing said first and said second elements into said bone, one of said two elements carrying a surgical suture, a suture catcher being arranged within that element of said two elements not carrying said suture, by means of which suture catcher said suture can be taken hold of, after said two elements have met, and said suture can be withdrawn via said element not carrying said suture by said suture catcher.

According to the invention, a separate suture catcher is provided in that element not carrying a suture. This has the effect that, after the two elements have met, the suture can be taken hold of by the suture catcher and can be withdrawn via the element in which the suture catcher is arranged, without any danger of the suture coming loose as it is withdrawn by the suture catcher.

The two elements remain in the bone in the position in which they have met. The operator uses the suture catcher to take hold of the suture that has been guided by the suture-carrying element to the element with the suture catcher. The element carrying the suture carries the latter in such a way that it is taken hold of by the suture catcher when the latter is maneuvered. The element comprising the suture catcher forcibly guides the latter into a position in which the suture is caught by the suture catcher. The suture can now be withdrawn from the bone by means of the suture catcher via the first element. This transfer of the suture can be carried out with a high success rate, an almost 100% success rate.

In a first basic approach, the suture catcher is arranged in the first element, and the suture is carried by the second element. The first element is driven into the bone first. Thereafter, the second, movable element that carries the surgical suture is driven into a hole in the bone until it meets the first element. After the two elements have met up, the suture is taken hold of by the suture catcher, arranged in the first element, and withdrawn via the first element. It is only then, i.e. when the free ends of the suture are secured outside the bone by the operator, that the two elements of the device are removed from the bone.

In a second basic approach, the suture is carried by the first element, and the suture catcher by contrast is arranged in the second element. The first element carrying the suture is driven into the hone first. The second element is likewise driven into the bone, specifically until it meets the first element. In this state, the suture is taken hold of by the suture catcher arranged in the second element and is withdrawn via the second, movable element.

In one embodiment of the invention, the first element is immovable and rod-shaped.

This measure has the advantage that, when the device according to the invention is applied to the desired site on the bone, this immovable first element, which is advantageously driven first into the bone, defines a positionally fixed point of engagement. The immovable element positioned in this way, which is rod-shaped, can easily be driven into the bone by the operator.

In another embodiment of the invention, a distal end of the first element is designed as a tip.

The advantage of this is that a rod-shaped element whose distal end is designed as a tip can be driven easily into the bone with minimal trauma.

In another embodiment of the invention, the suture catcher is arranged in the first, rod-shaped element.

This measure has the advantage that the suture catcher, like the first element, has a straight body, which is preferably made of a rigid material. This has the effect that, after the two elements have met up, the suture can be easily taken hold of by the suture catcher by way of a rectilinear displacement movement.

In another embodiment of the invention, a distal portion of the first element is provided with an opening.

This measure has the advantage that the second element can travel through this opening into the first element. This creates a point of intersection of the first element and the second element after the two elements have met. The point of intersection defines a transfer station for the suture. Such a design of the first element provides that the suture is brought to a transfer station at which it can be taken hold of by the suture catcher arranged in the first element. This also has the effect that the suture transfer can be carried out with a high success rate.

In another embodiment of the invention, a distal portion of the second element carrying the suture can be driven into the opening in the first element.

Such a design of the device according to the invention ensures that the suture carried by the distal portion of the second element is brought to the transfer station by means of the second element being driven into the opening in the first element. The suture catcher, which is arranged in the first, rod-shaped element, can thus securely take hold of the suture situated at the transfer station.

In another embodiment of the invention, the second element carries the suture at its distal portion in such a way that a suture portion of the suture is exposed and, after the elements have met, can be forcibly taken hold of by the suture catcher arranged in the first element.

Such a design of the device according to the invention means that the taking hold of or catching of the exposed suture portion of the suture by the suture catcher is not dependent on the dexterity or attentiveness of the person operating the device, since the suture catcher forcibly takes hold of the suture that has been brought to the transfer station. This avoids errors in taking hold of the suture. Consequently, the suture transfer can be carried out with a 100% success rate.

In another embodiment of the invention, the suture catcher can be introduced into the first element only in a defined position.

The advantage of this is that the positionally correct insertion of the suture catcher into the first element does not depend on the dexterity of the operator, because the correct insertion is predetermined by the design of the device according to the invention. In other words, the operator can insert the suture catcher into the first element only in such a way that the suture catcher forcibly takes hold of the suture that has been brought to the transfer station.

In another embodiment, a cavity for receiving the suture catcher is present in the first element.

By virtue of the fact that the suture catcher is received in the cavity, the suture catcher can be driven together with the first element into the bone without any danger of the suture catcher being damaged. Moreover, the first element can be driven first into the bone, and the suture catcher can thereafter be inserted in the first element.

In addition, the cavity in the first element ensures targeted guidance of the suture catcher, such that the suture catcher is guided to the transfer station where it securely takes hold of the suture.

In another embodiment of the invention, the cavity opens at its distal end into the opening in the first element.

This measure has the advantage that the suture catcher can protrude through the cavity and into the opening, which is arranged at the distal portion of the first element and which defines the transfer station. This ensures that the suture catcher securely takes hold of the exposed suture portion of the suture after the distal portion of the second element has been driven into the opening in the first element.

In another embodiment of the invention, the distal portion of the second element carrying the suture has a recess via which the exposed suture portion can be taken hold of by the suture catcher.

This once again has the advantage that the exposed suture portion of the suture can be easily and securely taken hold of by the suture catcher via the recess.

In another embodiment of the invention, the distal portion of the second element has a bore through which the suture passes and which intersects the recess.

This measure has the advantage that the exposed suture portion is arranged transversely with respect to the recess in the second element, such that the exposed suture portion of the suture can be taken hold of by the suture catcher via the recess.

In another embodiment of the invention, the recess is designed as a groove.

This measure has the advantage that the groove can be formed in the second element in a particularly simple production technique, as a result of which the cost of production of the device in this embodiment is reduced.

As an alternative to this, it is preferable to design the recess or the groove as an oblong hole.

This embodiment of the recess in the form of an oblong hole has the advantage that the exposed suture portion of the suture can be easily taken hold of through the recess. The suture catcher can first be guided through the oblong hole and then brought behind the suture portion, in order then to draw the latter upwards.

In another embodiment of the invention, the second element is curved.

This measure has the advantage that, by means of such a combination of a rod-shaped element together with a curved element, a device is created with which a channel approximately in the shape of a quarter circle is formed in the bone. This channel is therefore shorter than an approximately semicircular channel that is formed with the known device. This has the result that the bone is less adversely affected.

In another embodiment of the invention, the second element has a tip at its distal end.

This once again has the advantage that the curved element can be driven easily and with minimal trauma into a hole present in the bone.

In another embodiment of the invention, the second element can be guided along a guide path on the device.

This measure has the advantage that the second element can be guided along a predetermined path which is oriented in such a way that the distal portion of the second element travels into the opening in the first element. This ensures that the exposed suture portion is brought to the transfer station defined by the opening, such that it can be taken hold of by the suture catcher.

In another embodiment of the invention, the suture catcher has a catching slit for receiving the suture.

This measure has the advantage that after the suture has been taken hold of by the suture catcher, it has a secure seat in the catching slit during withdrawal of the suture via the first element. The suture can thus be withdrawn without any danger of its coming loose from the suture catcher.

In another embodiment of the invention, the suture catcher is provided with a tip whose end point is laterally offset with respect to a central longitudinal axis of the cavity in the first element.

This affords the advantage that the laterally offset tip of the suture catcher runs past the exposed suture portion as the suture is taken hold of. This ensures that the tip does not meet the suture, and the suture is thus not damaged by the tip.

Further features and advantages will become clear from the following description and from the attached drawing.

It will be appreciated that the aforementioned features and the features still to be explained below can be used not only in the cited combinations but also in other combinations or singly, without departing from the scope of the present invention.

### BRIEF DESCRIPTION OF THE DRAWINGS

The invention is described and explained in more detail below on the basis of selected illustrative embodiments and with reference to the attached drawings, in which:

FIG. 1 shows a side view of a device according to the invention in a starting position;

FIG. 2 shows a view comparable to the view in FIG. 1, after the two elements have met;

FIG. 3 shows an enlarged view of a distal portion of a first element and of a second element, and a side view of a suture catcher;

FIG. 4 shows an enlarged and partially cross-sectional view in which the distal portion of the second element is driven into an opening in the first element;

FIG. 5 shows an enlarged and partially cross-sectional side view after the two elements have met, and in which the suture catcher is guided into the first element;

FIG. 6 shows a view comparable to the view in FIG. 5, with the suture catcher meeting an exposed portion of the suture;

FIG. 7 shows a view comparable to the view in FIG. 5, with the suture fixed in a catching slit of the suture catcher;

FIG. 8 shows a situation during formation of a channel in a bone using the device according to the invention, where the first element is driven into the bone; and

FIG. 9 shows a view comparable to FIG. 8, where the second element is driven into the bone and the suture is withdrawn by the suture catcher via the first element.

### DETAILED DESCRIPTION OF A PREFERRED EMBODIMENT

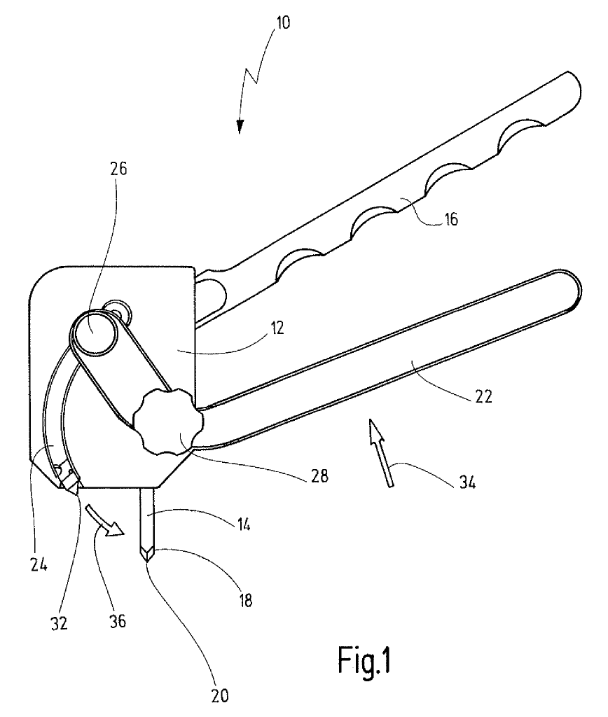

In FIGS. 1 to 9, a device for securing a surgical suture to a bone is designated in its entirety by reference number 10.

The device 10 according to the invention has a housing 12 to which a first element 14 and a grip 16 are fixedly connected.

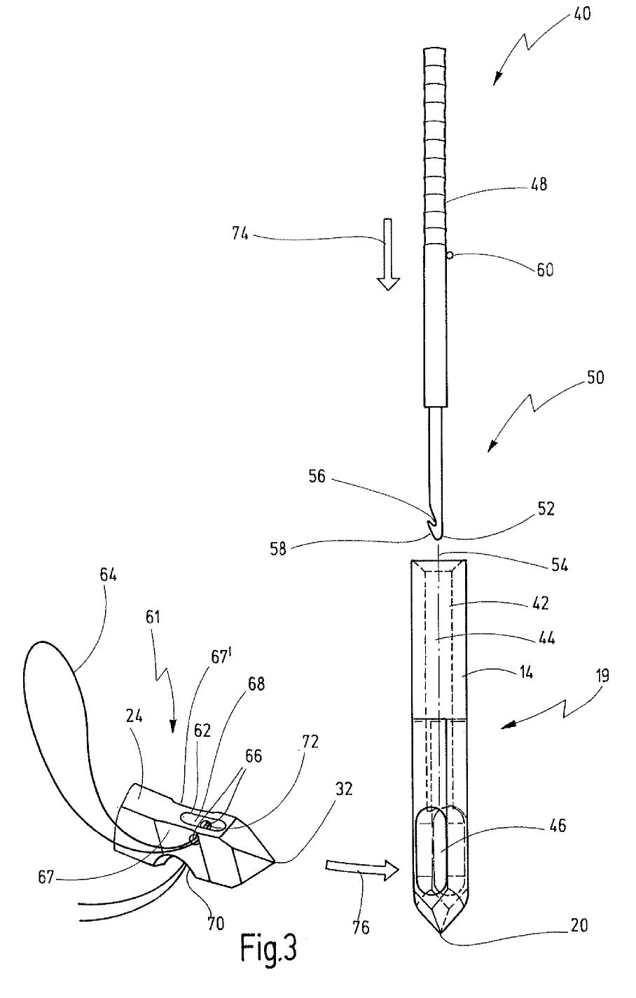

The first element 14 is immovable and rod-shaped. Moreover, the first element 14 is hollow in order to receive a suture catcher, as will be described in more detail below with reference to FIG. 3.

A distal end 18 of the first element 14 is designed as a tip 20, such that the rod-shaped element 14 can be driven easily and with minimal trauma into a bone.

The device 10 according to the invention further comprises a movable lever 22, on which a second, movable element 24 is mounted by way of a journal 26. The movable lever 22 is secured on the housing 12 by means of a nut 28. By loosening the nut 28, the lever 22, with the second element 24 mounted on the lever, can be removed from the housing 12, such that the device 10 can be thoroughly cleaned.

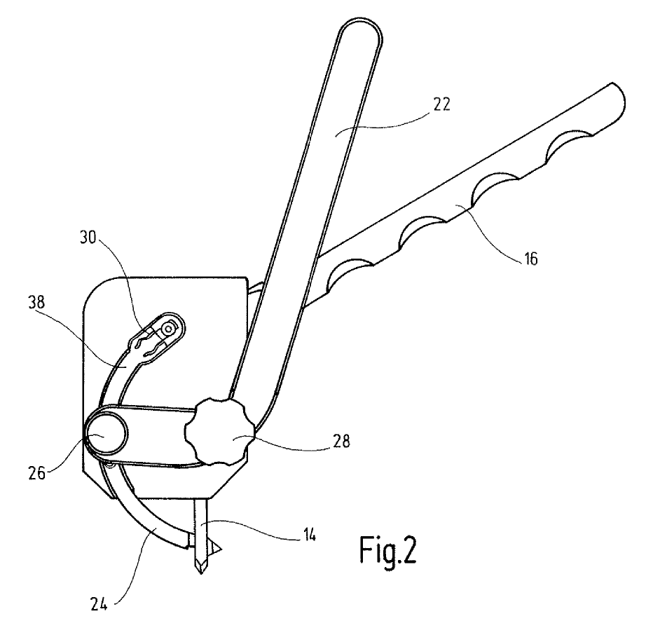

The lever 22 is held in the starting position shown in FIG. 1 by means of a clamp 30, which can bee seen from the view in FIG. 2.

The second element 24 has a curved shape and, at its distal end, has a tip 32 that makes it easier to drive the element 24 into the bone.

To bring the device according to the invention from the starting position shown in FIG. 1 to the second position shown in FIG. 2, the movable lever 22 is pulled in a direction indicated by an arrow 34. The movement of the lever 22 causes a movement of the second element 24 connected to the lever 22, as is indicated by an arrow 36. As the lever 22 is pulled, the second element 24 is moved along a guide path 38 which is formed in the housing 1Z and which defines the travel of the second element 24. The guide path 38 is oriented in such a way that the second element 24 meets the first element 14. Such a situation can be seen from FIG. 2.

FIG. 3 is an enlarged view of a distal portion 19 of the first element 14 and of a distal portion 61 of the second element 24. A suture catcher 40 can also be seen.

It will be seen from this figure that the first element 14 has a cavity 42 for receiving the suture catcher 40. The cavity 42 is designed as a channel 44 extending in the axial direction. At its distal end, the channel 44 opens into an opening 46 in the form of an oblong hole arranged on the distal portion 19 of the first element 14, such that the suture catcher 40 can travel through the channel 44 into the opening 46.

FIG. 3 also shows the distal portion 61 of the second element 24, which carries a suture 64.

The distal portion 61 has a bore 66 through which the suture 64 passes. At its bottom, the distal portion 61 of the second element 24 has an indentation 70. The indentation 70 serves to ensure that the suture 64 threaded through the bore 66 can be drawn back again.

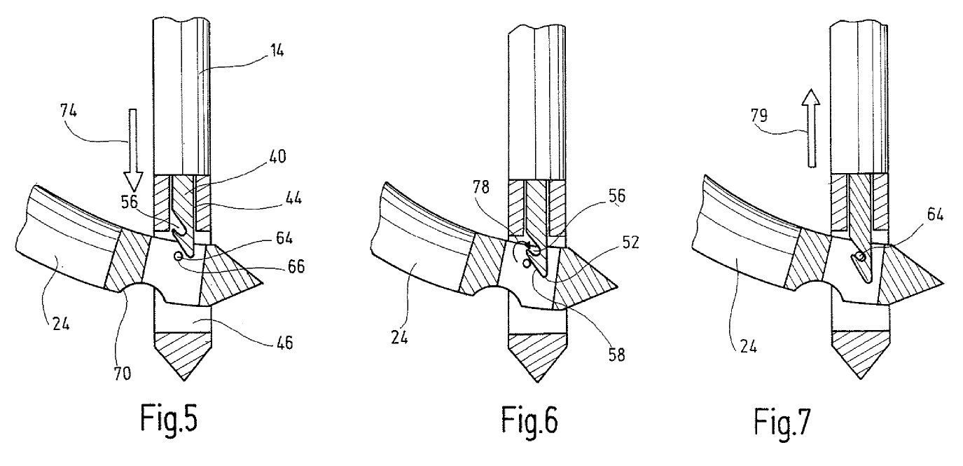

Moreover, the distal portion 61 of the second element 24 has a recess 62 via which an exposed suture portion 72 of the suture 64 can be taken hold of by the suture catcher 40, as will be described in more detail below with reference to FIGS. 5 to 7.

The recess 62 is designed as an oblong hole 68.

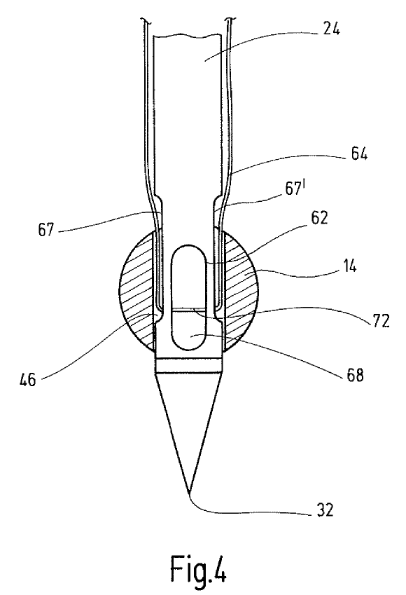

The bore 66 through which the suture 64 is guided intersects the recess 62 approximately centrally. Thus the exposed suture portion 72 of the suture 64 is arranged transversely with respect to the recess 62, as will be seen in particular from the view in FIG. 4.

To both sides of the bore 66 there are external indentations 67, 67′ that create space for the suture 64, such that the latter is not damaged by the edges of the opening 46 in the distal portion 19 of the first element 14.

FIG. 3 also shows the suture catcher 40, which has a rod-shaped body 48. The body 48 has a distal portion 50, which has a smaller diameter than the rest of the body 48. The diameter of the body 48 of the suture catcher 40 is adapted to cavity 42 designed as a channel 44 in the first element 14.

The suture catcher 40 also has a tip 52, which is laterally offset in relation to a central longitudinal axis 54 of the cavity 42 in the first element 14, the tip 52 being laterally offset to the extent that the tip 52 comes to lie near the wall of the cavity 42.

Moreover, the distal portion 50 of the suture catcher 40 is provided with an oblique catching slit 56 that serves to receive the suture 64. The catching slit 56 is open at its end directed away from the tip 52, such that the suture 64 taken hold of by the suture catcher 40 is securely fixed in the catching slit 56 when it is withdrawn via the first element 14.

The tip 52 also has an inclined flank 58, such that the suture 64 can slide along the flank 58 into the catching slit 56.

The suture catcher 40 comprises a pin 60 on the body 48. The pin 60 defines the position in which the suture catcher 40 can be guided into the cavity 42 of the first element 14.

As has already been described, pulling the lever 22 causes the second element 24 to move along the guide path 38, in a direction indicated by an arrow 76, until the distal portion 61 of the second element 24 carrying the suture 64 passes into the opening 46 in the first element 14.

After the two elements 14, 24 have met, i.e. when the distal portion 61 of the second element 24 is driven into the opening 46, the exposed suture portion 72 of the suture 64 can be taken hold of by the suture catcher 40, as will now be described with reference to FIGS. 5 to 7.

To take hold of the suture 64, the suture catcher 40 is guided into the channel 44 in the first element 14 (see arrow 74) after the distal portion 61 of the second element 24 has been driven into the opening 46 in the first element. It should be noted that the suture catcher 40 can be introduced into the channel 44 in a position predetermined by the pin 60. The suture catcher is then displaced axially until the flank 58 meets the exposed suture portion 72 of the suture 64, as can be seen from FIG. 6.

The fact that the tip 52 of the suture catcher 40 is laterally offset means that it does not meet the suture 64 but instead runs past the latter. This ensures that the suture 64 is not damaged.

Upon further displacement of the suture catcher 40, the suture 64 slides along the inclined flank 58 and enters the catching slit 56 (see arrow 78). This situation is shown in FIG. 7.

After the suture 64 has been caught in the catching slit 56, the suture catcher 40 together with the suture 64 is withdrawn via the first element 14 (see arrow 79).

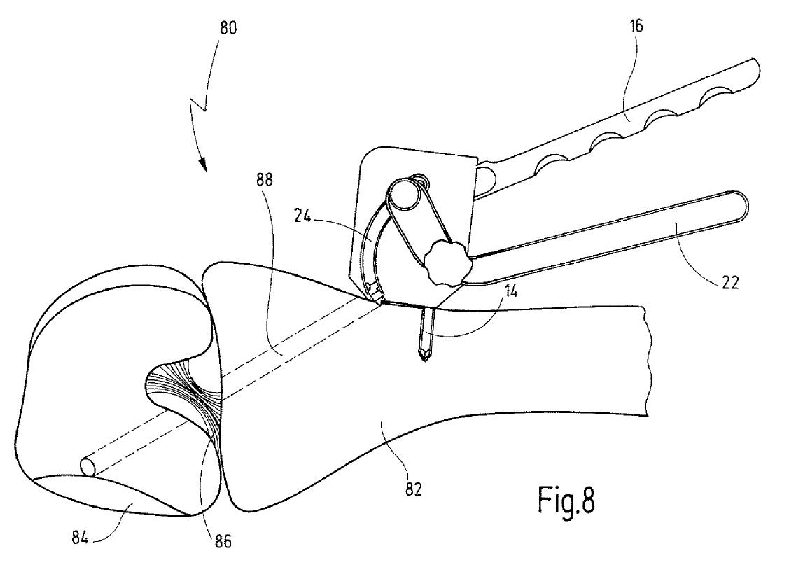

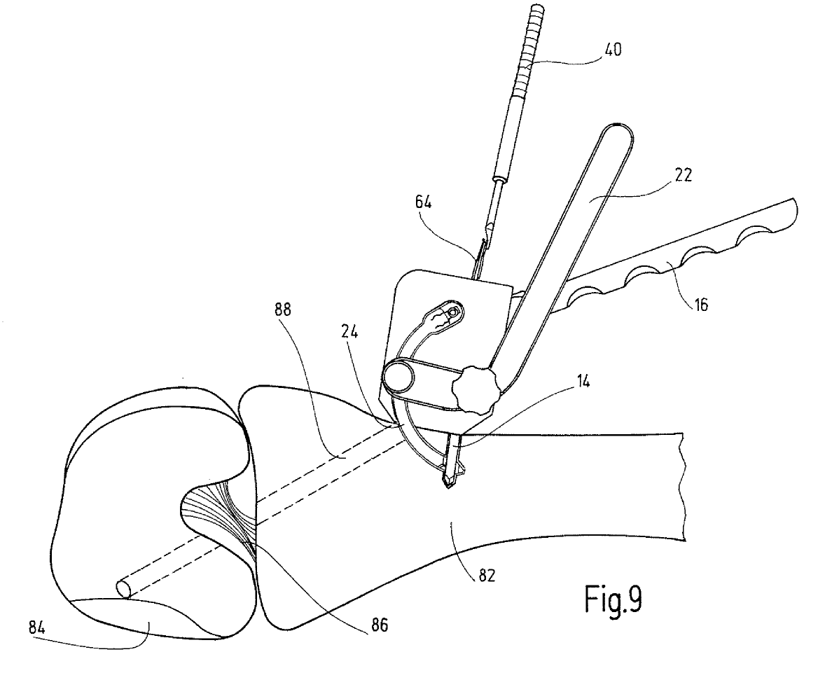

A possible use of the device 10 according to the invention will be explained briefly with reference to FIGS. 8 and 9.

FIG. 8 is an extremely schematic representation of a human knee joint 80.

The knee joint 80 is a connecting joint between the tibia (shin bone) 82 and the femur (thigh bone) 84. The thigh bone is angled rearwards from the plane of the drawing. At the centre of the human knee there are two intersecting ligaments extending from the tibia 82 to the femur 84, namely the posterior cruciate ligament 86, and the anterior cruciate ligament, which is not shown here and is intended to be replaced by an implant.

In reconstruction of a torn anterior cruciate ligament, an implant (not shown here) is introduced into a bore 88, which has been drilled beforehand in the bone, and is secured to the corresponding bone or to the body.

In this surgical procedure shown in FIG. 8, a suture 64 (not shown here) holding the implant is intended to be secured to the tibia 82.

The device 10 according to the invention is used for this purpose. First, the distal portion 61 of the second element 24 is fitted with the suture 64 of the implant, specifically in the manner shown in FIG. 3. In the starting position, the device 10 according to the invention is mounted on the tibia 82 in such a way that the tip 32 of the second element 24 is applied at the tibial bore 88.

The first element 14 is then driven into the tibia 82. The movable lever 22 is pivoted, as a result of which the curved element 24 is driven into the tibia 82.

After the elements 14, 24 have met, the suture catcher 40 is guided into the cavity 42 designed as a channel 44 in the first element 14, in order to take hold of the exposed suture portion 72 of the suture 64 carried by the second element 24. The suture 64 is taken hold of exactly in the manner described with reference to FIGS. 5 to 7.

Thereafter, the suture catcher 40 together with the suture 64 is withdrawn via the first element 14. This situation is shown in FIG. 9.

Finally, the second element 24 is returned to the starting position by moving the movable lever 22, and the first element 14 is then withdrawn from the tibia 82.

With the suture 64 secured on the tibia 82, the suture (not shown here) holding the implant can be fixed on the tibia 82.

What is claimed is:

1. A device for securing a surgical suture to a bone, comprising
a first element, which can be introduced into a bone,
a second, movable element, which can be introduced into said bone in such a way that it meets said first element,
a channel being formed in said bone as a result of introducing said two elements into said bone,
one of said two elements carries a surgical suture,
a suture catcher is arranged within that element of said two elements not carrying said suture, by means of which suture catcher, said suture can be taken hold of, after said two elements have met, and said suture can be withdrawn via said element not carrying said suture by said suture catcher.

2. The device of claim 1, wherein said first element is immovable and rod shaped.

3. The device of claim 1, wherein a distal end of said first element is designed as a tip.

4. The device of claim 2, wherein said suture catcher is arranged in said first, rod-shaped element.

5. The device of claim 4, wherein a distal portion of said first element is provided with an opening.

6. The device of claim 5, wherein a distal portion of said second element carrying said suture can be driven into said opening in said first element.

7. The device of claim 6, wherein said second element carries said suture at its distal portion in such a way that a suture portion of said suture is exposed and, after said two elements have met, can be forcibly taken hold of by said suture catcher arranged in the first element.

8. The device of claim 4, wherein said suture catcher can be introduced into said first element only in a defined position.

9. The device of claim 4, wherein a cavity for receiving said suture catcher is present in said first element.

10. The device of claim 9, wherein said cavity opens at its distal end into said opening in said first element.

11. The device of claim 7, wherein said distal portion of said second element carrying said suture has a recess via which said exposed suture portion can be taken hold of by said suture catcher.

12. The device of claim 11, wherein said distal portion of said second element has a bore through which said suture passes and which bore intersects said recess.

13. The device of claim 12, wherein said recess is designed as a groove.

14. The device of claim 13, wherein said groove is designed as an oblong hole.

15. The device of claim 1, wherein said second element is curved.

16. The device of claim 1, wherein said second element has a tip at its distal end.

17. The device of claim 1, wherein said second element can be guided along a guide path on said device.

18. The device of claim 1, wherein said suture catcher has a catching slit for receiving said suture.

19. The device of claim 1, wherein said suture catcher is arranged in said first, rod-shaped element, a cavity for receiving said suture catcher is present in said first element, and wherein said suture catcher is provided with a tip, whose end point is laterally offset with respect to a central longitudinal axis of said cavity in said first element.
